(19) United States
(12) Patent Application Publication
(10) Pub. No.: US 2011/0077527 A1
(43) Pub. Date: Mar. 31, 2011

(54) SELF-COOLING GEL SUBSTRATE FOR TEMPERATURE DIFFERENTIATED IMAGING

(76) Inventors: SeungRim Yang, Seongnam-si (KR); JinHo Ryu, SuWon-si (KR); MiYun Kwon, Yongln-Si (KR); WanDuk Lee, Seoul (KR); John Gavin MacDonald, Decatur, GA (US); Jaeho R. Kim, Roswell, GA (US)

(21) Appl. No.: 12/571,119

(22) Filed: Sep. 30, 2009

(51) Int. Cl. A61B 6/00; B41M 5/40

(52) U.S. Cl. 600/474; 428/32.6

(57) ABSTRACT

A device and clinical techniques that can help healthcare workers locate and image the general outlines of blood vessels that are located either under or near to tissue or skin surfaces are described. The device involves a substrate formed from a self-cooling polymeric matrix that incorporates at least a thermo-chromic colorant, and can provide a means to distinguish between different temperature regions when applied to a heat-emitting object or body. The device can be used in certain therapeutic or healthcare-related applications.

### FIELD OF INVENTION

The present invention relates to a device that can be used in certain therapeutic or healthcare-related applications. In particular, the present invention pertains to a self-cooling polymeric gel-pad that incorporates at least a thermo-chromic colorant, and can provide a way to distinguish between different temperature regions when applied to a heat-emitting object or body.

### BACKGROUND

Common medical tests or procedures performed on patients often involve obtaining and analyzing a sample of a patient's blood or the infusion of fluid into a patient. These procedures involve the insertion of a needle into the patient's blood vessels, typically a vein. Of course, to puncture the vein of the patient, the vein must first be located. The location of the vein is not particularly difficult if it can be visually located or palpated. To enhance the probability of visual sighting or feeling, a tourniquet (e.g., an elastic strap) is often applied between the targeted area for insertion of the needle and the patient's heart. For examples, a tourniquet could be applied around the upper arm of a patient when the location for insertion of a needle is near the hand or elbow. This produces a differential in the pressure of the blood being conducted by the veins. The human body responds to such a pressure differential by enlarging the veins in an attempt to provide a conduction path of less resistance. The enlarging of the veins makes them more prominent and therefore increases the probability that one of the veins can be located by viewing or feeling the arm of the patient. Unfortunately, the procedure for enlarging the veins is not always successful. For instance, because the vein is generally dark in color, it is even more difficult to sight a vein in the arm of a person having a dark colored pigment in his skin. Other characteristics of the patient that make it particularly difficult to sight or feel a vein are associated with small children, obesity, and old age. These characteristics generally mean that the vein is significantly recessed from the skin and therefore particularly difficult to see or feel.

Various techniques have thus been developed to aid in the location of veins. Medical persons and clinical researcher have had an interest in thermal imaging of the venous or general circulatory system of warm-blooded animals. Use of thermochromic ink solutions suggests certain benefits for simple and efficient techniques for identifying the location of veins just under the skin surface. One such technique relies upon the fact that the temperature of the skin in proximity to a vein is generally greater than the temperature of the remaining portions of the skin. To detect the higher temperatures of the skin adjacent to the vein, liquid crystal materials have been employed that undergo a color change at the desired temperature. To improve color contrast, the liquid crystals are commonly applied to and viewed against a black background that serves to absorb the transmitted light. U.S. Pat. No. 3,998,210 to Nosari, for example, describes the use of encapsulated liquid crystals in a laminated article that includes a black background for locating veins in the body. Still another technique for enhancing the color contrast is described in U.S. Pat. No. 4,175,543 to Suzuki et al., which involves cooling the skin with a cold pack before or after application of microencapsulated liquid crystals to produce a greater temperature gradient between the skin surface directly over the vein and adjacent areas of the skin. This temperature gradient is said to provide a sharper delineation of the vein for identification. One problem with the conventional vein identification methods, however, is that the liquid crystals employed generally have a low color density, poor color selectivity and are expensive. Further, the methods involved are too complex in that they often involve multiple steps to be performed by the user, such as color contrast, cooling, and so forth.

As such, a need currently exists for a simple, efficient, and effective method for rapidly identifying the presence or absence of blood vessels.

### SUMMARY OF THE INVENTION

The present invention pertains, in part, to a thermal-imaging article or aid that can help healthcare workers more easily visualize the blood vessel network that is located is near the surface of a patient's skin. More particularly, the invention describes a temperature-sensitive substrate that can change color relative to the level of heat transmitted from different areas or regions of a heat-emitting body or substrate, such as a human or mammalian body. The temperature-sensitive substrate can be a membrane, film sheet or gel-pad that is formed from a self-cooling polymeric gel matrix having at least a thermochromic colorant that is either admixed within the gel matrix or formed as a layer on one of its major surfaces. The gel matrix manifests a change of optical characteristics or physical properties, such as color, opacity and/or volume around certain programmed temperature ranges (e.g., color change temperature of a thermochromic colorant, lower critical solution temperature (LCST) of polymer matrix). This observable change in appearance is relatively fast and easily detectable, which makes it suitable for use in the visual indication of temperature differences or changes. The colorant coated surface (i.e., first major surface) is to contact the heat-emitting body; while, the uncoated surface (i.e., second major surface) is oriented away from the heat-emitting body for a user to observe any color changes that may arise from the thermochromic colorant. The amount of color change can be significant and observable by the naked eye, with ΔE values in a range of about 15 or 20 to about 60 or 65. The temperature-sensitive substrate can take the form of a variety of different planar geometries. Between the two major surfaces, the substrate can have a thickness from about 0.1 or 0.2 mm up to about 7 or 8 mm. A plurality of pores are distributed in either a regular predetermined pattern or randomly over the substrate, and each of the pores traverses from the first surface to the second surface, or in other words from one side of the substrate to the other. The thermo-chromic colorant can be one of the following: microcapsules include a proton-accepting chromogen, or liquid crystal or fatty acid derivatives of cholesterol system.

For health care-related purposes, the temperature sensitive substrate can be self-tacking when applied against mammalian skin, and may include an antimicrobial or anti-pain analgesic agent, or a combination thereof that is topically or locally released when applied against mammalian skin. Alternatively, other means (e.g., adhesives, rubber bands, elastics) could be employed for ensuring intimate skin contact even during movement of the patient. These active antimicrobial, antiseptic or analgesic agents may be incorporated either within the polymeric gel matrix or as a coating on at least one of the major substrate surfaces. The thermochromic colorant should have a color-change sensitivity at a temperature in a range between about 35.0° C. to about 40.5° C., which includes the normal human body temperature range (i.e., 37.0° C. (98.6° F.) commonly accepted average core body temperature, or alternatively, 36.8±0.7° C. (98.2±1.3° F.) average oral temperature). The self-cooling polymer gel matrix (e.g., a hydrogel) is designed to evaporate a cooling agent (e.g., water) when applied against a source of heat.

As described above, the thermo-sensitive colorants in the temperature sensitive substrate can manifest a change in the color or opacity of the gel pad when placed against a patient's skin. The pad is applied in contact with the surface of the skin directly over the general location of an artery or vein. The relative changes in skin temperature between areas of skin near a major blood vessel, which typically is warmer, versus areas of skin not near a major blood vessel in the surrounding tissues outlines the location of blood vessel in the skin that can serve as a target for insertion of a needle. A self-cooling polymeric gel lowers the local temperature of the skin for a greater temperature gradient between the skin surface directly over the blood vessels and their adjacent tissues. In the case of a hydrogel matrix, for instance, the present invention contributes developing a sharper delineation of blood vessels for better imaging by means of the continuous cooling evaporation of water from the hydrogel. Further since a hydrogel can maintain the temperature gradient for longer period, this can help enhance better visual distinction of vein imaging over a time window for a healthcare worker to work.

In another aspect, the present invention pertains to a method for detecting and thermal-imaging body regions with greater localized temperatures than surrounding tissues of a patient. The method involves providing a self-cooling polymer gel substrate containing a thermo-chromic colorant; applying said gel substrate to a heat-emitting mammalian body; observing a color contrast that develops from a temperature gradient between areas of said gel substrate that cover warmer body regions and adjacent cooler body regions. For instance, one may apply the self-cooling polymer gel substrate to areas of the mammalian body where subdural arterial or venous patterns are located near the tissue or skin surface (e.g., within about 1-3 cm). For instance, an application of the present invention can be adapted to locate target areas for application of a skin patch for drug delivery.

Alternatively, the invention can be also a method for locating a blood vessel. The method having the steps of providing a self-cooling polymeric substrate containing a thermochromic colorant that changes color at a temperature in a range between about 35.0° C. to about 40.5° C.; placing the polymeric substrate against bare skin of a portion of a body over which blood vessels may be accessed; observing a color contrast develop that arises from a temperature gradient between areas of the gel substrate that cover body regions that are warmer that their adjacent cooler body regions; and inserting a needle or cannula into a blood vessel that is thermal-imaged by the polymeric substrate. The substrate can prolong the difference in relative temperature gradient between blood vessels and surrounding tissues for up to about 6 or 7 minutes and enhance visual-image contrast.

Additional features and advantages of the present device and methods will be described in the following detailed description. It is understood that the foregoing general description and the following details description and examples are merely representative of the invention, and are intended to provide an overview for understanding the invention as claimed.

### BRIEF DESCRIPTION OF FIGURES

FIGS. 1A and 1B are schematic representations of gel substrates according to two embodiments of the present invention. FIG. 1A depicts an embodiment in which thermochromic colorants are dispersed evenly throughout the polymer gel matrix of the substrate, and FIG. 1B shows an embodiment in which the thermochromic colorant is either applied as a layer or thin film on a lower surface of the gel substrate.

FIG. 2A shows a schematic overview of a gel substrate perforated with a plurality of small holes or pores.

FIG. 2B is an enlarged, lateral, cross-sectional view of a portion of the gel substrate of FIG. 2A, showing a number of channels or pores that traverse the gel substrate. A tip of a hypodermic needle is depicted as about to be inserted into underlying skin through one of the pores.

FIG. 3 is an illustration of a human arm and hand with two temperature-sensitive, self-cooling gel substrates according to the present invention; one applied to the dorsal portion of the hand and another to the inner bend of the arm.

FIG. 4 is a photograph showing a self-cooling gel substrate according to the present invention, applied to the dorsal portion of the human hand, such as illustrated in FIG. 3, generating a temperature differentiated imaging of blood vessels immediately underlying the skin.

FIG. 5 is a photograph showing the gel substrate according to the present invention, applied to the inside crook of the human arm, such as illustrated in FIG. 3, and showing a temperature differentiated image of blood vessels immediately underlying the skin.

FIG. 6 is a representation of a gel substrate according to the present invention, applied to a portion of a human forearm, and showing temperature differentiated image of underlying blood vessels.

FIG. 7 is another image of a self-cooling gel-substrate applied to the back of a human hand (FIG. 1B type).

FIG. 8A-8D are a sequence of photographs showing a comparative example of the development of a temperature differentiated image of a blood vessels under the skin of the back of a human hand when a thermochromic ink alone, without application of a self-cooling substrate, is applied directly to the skin.

FIG. 9A-9G are a sequence of photographs showing the development of a thermal image of blood vessel pattern on the back of a human hand that is covered with a gel substrate according to an embodiment of the present invention. The thermal image develops over about 1-2 minutes from FIG. 8A to FIG. 8G to reveal the outlines of the subdural network of blood vessels.

### DETAILED DESCRIPTION OF THE INVENTION

Generally, the present invention describes a device and clinical techniques that can help healthcare workers locate and image the general outlines of superficial blood vessel networks (i.e., within about 0.2-0.5 cm, or about 1-2 cm) that are located either under or near to tissue or skin surfaces. For instance, the invention can be used to help healthcare workers more easily withdraw blood or insert an intravenous line. Conversely, the device can help healthcare workers avoid blood vessels, or locate areas that are largely free from large veins or arteries. In some embodiments, if properly sterilized, the invention could also be applied against the surface of internal tissues or organs during surgery, to help the surgeon to either void or target sensitive areas where higher localizes temperatures can be imaged or visual observed by the naked eye (e.g., cancer tissues where blood vessel growth, density, and blood flow may be abnormally high). The body region that is the focus for imaging can have a temperature difference of as low as about 0.1° C. or 0.2° C. to about 2° C. or 3° C. or greater than its surrounding tissues.

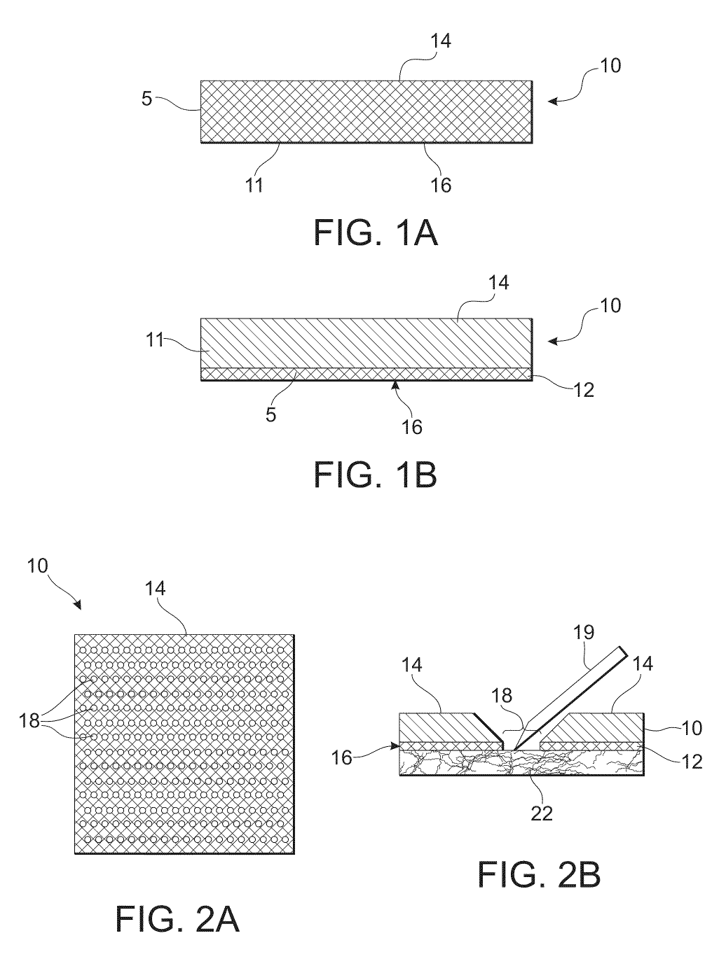

The device involves a substrate 10 formed of a self-cooling polymer gel matrix having at least a thermochromic colorant or dye 5 that is either admixed within the polymer matrix 11 or formed as a film or coating 12 on one of the major surfaces 14, 16 of the substrate 10, such as illustrated in FIGS. 1A and 1B respectively. The thermo-chromic colorant 5 changes color in response to the relative amount of heat emitted from the area covered under the gel substrate to produce a visually distinct contrast in color between a hotter area and a cooler area. A plurality of pores 18 is arranged in a pattern over the surface 14 of the substrate 10 such as shown in FIG. 2A. The pores can be randomly distributed or arranged in a regular, evenly spaced pattern. The distance between each pore, on center, should not exceed, but be about the same dimension as the average width or diameter of each pore. Each pore 18 extends through the gel substrate 10 from a first major surface 14 to a second major surface 16, as depicted in FIG. 2B. Although not shown to scale, each pore 18 is adapted to accommodate the diameter or width of a hypodermic syringe needle 19, so that a user can insert the needle or cannula along or through a pore to access the patient's skin 22 underneath the temperature-sensitive substrate 10 without needing to penetrate through the polymeric matrix 11 of the substrate when making an injection or drawing blood from a blood vessel. The pores can have a width dimension of about 0.05 or 0.07 mm up to about 1 mm, typically about 0.1 or 0.2 mm to 0.5 mm.

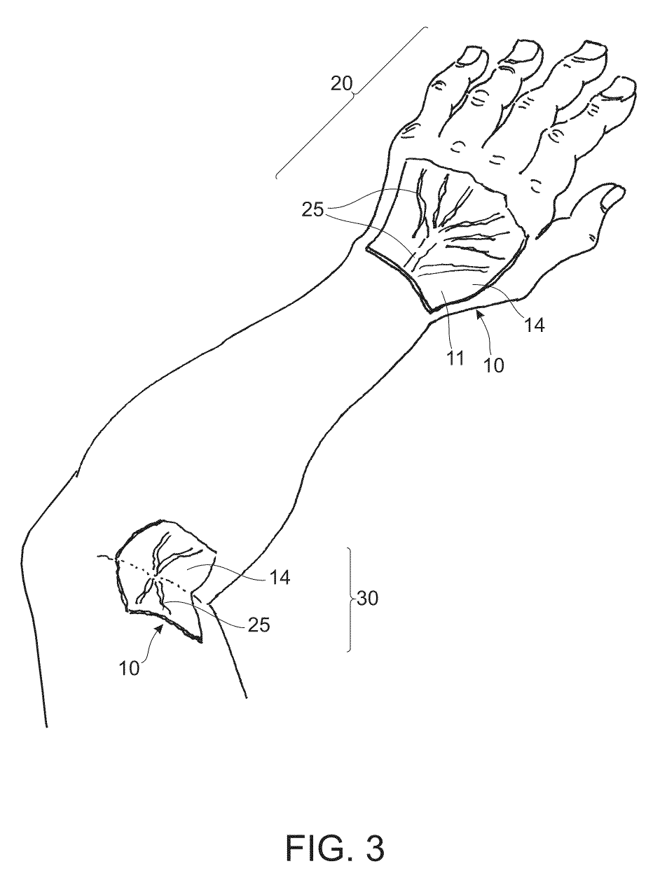

The self-cooling polymeric gel matrix can be made into a relatively thin pad (e.g., about 0.25-0.5 or 0.75 mm, 1 or 2-3 mm, or up to about 5-6 or 7 mm in thickness), having a thermo-sensitive colorant or dye that is applied against an area of the body where blood vessels rise close to the surface of the skin (e.g., neck/throat, back of the hands, inside crook of the arm or on the forearm, tops of feet or along the legs). FIG. 3 shows two self-cooling substrates 10 placed over the dorsal area of a human hand 20 and in the crook area of the arm 30 to show temperature differentiated images 25 of underlying blood vessels of each area. As adapted to better fit against these kinds of anatomical parts, the temperature-sensitive substrate may take the form of different planar geometric shapes without limitation. For instance, the substrate can be a square or other rectilinear forms, circular or elliptical shapes, bi-lobal or hour-glass-like forms, convex and/or concave sided irregular shapes, or forms that have a major axis and a minor axis that are largely orthogonal to each other. Typically for use with humans, square or rectilinear, circular or elliptical, and bi-lobal forms can be employed, and have dimensions of between about 3 cm, 4 cm or 5 cm up to about 7 cm, 8 cm or 10 cm along a side, diameter or major longitudinal axis, respectively. For other mammalian species, the practical dimensions may vary from about 1 cm or 2 cm up to about 20 cm or 30 cm or larger along a side, diameter or major axis, depending on the size of the animal. The particular shape or form is inconsequential as long as the substrate can attach the body relatively securely.

According to an embodiment, the self-cooling gel substrate can be formed from a hydrogel matrix having a thin temperature sensing layer with thermochromic colorants on one side of the hydrogel pad. The temperature sensing layer of the gel pad can be place in direct contact with mammalian skin. Desirably the colorant has a temperature sensitive activity range that is compatible with average human body temperature (i.e., ˜98.2-98.6° F.). Typically, the average oral temperature for healthy adults had is about 37.0° C. (98.6° F.), while normal ranges may vary from about 36.1° C. (97.0° F.) to about 37.8° C. (100.0° F.). Alternatively in another embodiment, variants of temperature sensitive colorant that are more adapted to the body temperature of infants and small children can also be incorporated.

The substrate in the embodiment according to FIG. 1A, in which the thermochromic dye or colorant is admixed in the substrate's polymeric matrix, the substrate may have a composition that includes a gel, a pigment, and water in a ratio that may range from 2-10:0.5-5:50-200 in terms of total weight percentage. In certain desirable embodiments, the ratio of gel: pigment:water can be, for example, 2:1:100, 3:1:160, 3:2:150, or 4:2:200 by weight. In the embodiment according to FIG. 1B, where the colorant is a separate layer or film coating the gel substrate, the ratio of pigment:water may range from 0.25-10:100 by weight. In certain desirable embodiments, the ratio of pigment:water can be 0.5-5:100 by weight. However, the weight percentage of the polymer compositional amount (not gel) is dependent on the kind of polymer incorporated in the composition; because, the amount of polymer that can be incorporated to achieve certain desired physical properties, such as tensile strength and elasticity of the gel substrate may vary for each kind of polymer material used. For example, 0.5˜5 wt % of agarose (e.g., 2 wt % being a desirable amount) in water can provide good physical properties for the gel substrate; while 5˜20 wt % of acrylamide (e.g., 10 wt %) can give similarly good properties.

Hydrogel (also referred to as “Aquagel”) is a network of polymer chains that are water-insoluble, sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. This makes them an ideal candidate to adhere closely to the contours of skin for the vein imaging applications. The structure of a hydrogel consists of a solid three-dimensional network that spans the volume of a liquid medium. This internal network structure may result from physical or chemical bonds, as well as crystallites or other junctions that remain intact within the extending fluid. Virtually any fluid can be used as an extender including water (hydrogels), oil (organogel). Both by weight and volume, gels are mostly liquid in composition and thus exhibit densities similar to those of their constituent liquids. The composition of a hydrogel may include: polyvinyl alcohol, sodium polyacrylate, and acrylate polymers and/or copolymers with an abundance of hydrophilic groups. Natural hydrogel materials, for instance, may include agarose, methylcellulose, hylaronan, and other naturally derived polymers.

During the period of use, the hydrogel matrix can lower and control the relative skin temperature, as continuous water-based evaporative cooling from the hydrogel enhances a temperature gradient between areas of the skin surface either directly over or adjacent to a blood vessel. The hydrogel provides an enthalpy of vaporization that is low enough to provide cooling to the skin. Generally speaking, the cooling agents have a latent heat of vaporization of about 45 kJ/mole or less, in some embodiments about 40 kJ/mole or less, and in some embodiments, from about 5 to about 39 kJ/mole. The cooling effect of the gel can prolong the effective time window for better image contrast of the temperature gradient. Also, this will allow for a greater color contrast and a sharper delineation of the warmer blood vessel against the cooler surrounding tissues. The blood vessel can be identified also immediately. Typically, the image begins to manifest within one or two minutes of applying the substrate against the skin surface.

According to an example of an embodiment, such as depicted in FIG. 1B, the thin temperature sensing layer is applied on the skin-contacting side of the gel substrate to promotes more greater sensitivity to temperatures and generate higher visual contrast. In some case, the gel sheet can be perforated, such as in FIGS. 2A and 2B with an array of small pores or holes that enable a hypodermic needle to penetrate through the gel sheet without causing an occlusion in the channel of the needle when inserted into the patient. The surface of the gel pad can be marked with a pen or other writing instrument to trace the imaged blood vessel network. As long as the gel substrate is not moved from its original location on the patient, this can help the healthcare worker easily locate underlying blood vessel channels even when the temperature of the blood vessel and surrounding tissues equilibrate and after the actual thermal generates image has becoming less distinct. Using a mold having a plurality of raised pins, bumps, or ridges, regularly or randomly spaced, one can create the pores when casting the gel pad.

In comparison, other approaches for visualizing blood vessels, such as applying a thermochromic ink solution alone directly on a skin surface, have not been as successful and have shown several disadvantages. First, one would need to wait for the applied solution to dry sufficiently before working. Second, one would need to prepare the skin to achieve a relatively cooler temperature than the warmer blood vessels before and after application of the ink solution so as to maximize the temperature gradient. Otherwise one can have less visual resolution between the blood vessel and the surrounding tissue. Third, after application, depending on the ambient environmental conditions, the ink solution will tend to acclimate shortly to an equilibrium temperature of the skin. In a short time after applying the thermochromic ink, the veins tended to appear thicker than their real dimensions due to the relatively low temperature gradient difference. Because of the tendency of such approaches to lose resolution when the temperature gradient between the skin surface directly over the vein and adjacent areas of the skin lessen as the skin recovers in short time, so the identification of vein eventually disappears. This phenomenon permits only a relatively short window of time (typically less than 1 or 2 minutes) in which the healthcare worker can operate before the sharp contrast of the temperature gradient between skin and veins begins to equilibrate and disappear. The thermochromic colorant can change color in a zoned area.

The temperature differentiated visual color contrast in the substrate can be characterized objectively. The shift from an initial hue or color to a different one can be characterized in terms of a ΔE value that signifies how easily observable the color change is. Subtle or slight distinctions in shades or hues of color can be difficult to detect. For a trained observer, color distinctions are detectable to a naked eye at a threshold ΔE value of about 3. For more common observers, visual differences or changes in color become detectable at ΔE of about 5 or 6. Hence, the optical or color indicative mechanism according to the present invention exhibits a ΔE value greater than three (>3), desirably greater than or equal to five (≧5), and more desirably greater than or equal to ten (≧10). In some instances the contrast ΔE values can range from about 12-15 or 20 up to about 70 or 80-85, inclusive. Typically, the ΔE value is about 20-40 or 50, up to about 60-65.

In the present invention, the hydrogel matrix either alone or combined desirably with a thermo-sensitive colorant in the pad placed can prolong the difference in temperature gradient and enhance the visual contrast image when placed against bare skin surface. This prolonged period can extend up to about 5-6 or 7 minutes. Typically, the clearest heat differential image resolution of sub-dermal blood vessels is between about 20-30 or 45 seconds to about 2-3 or 4 minutes. An optimal period of high visual contrast should last between about 1.5 or 2 minutes to about 4 or 5 minutes; this prolonged period affords a healthcare worker a sufficient window to effectively identify and perform clinical functions such as draw blood or insert intravenous (IV) feeds.

Since the thermo-chromic colorant or ink is to be applied against the skin of a patient (e.g., human or animal), one would desired that the thickness of the resulting coating on the hydrogel substrate is relatively small so as to enhance the detection sensitivity. For example, the thickness may range from about 0.01 millimeters to about 5 millimeters, in some embodiments, from about 0.01 millimeters to about 3 millimeters, and in some embodiments, from about 0.1 millimeters to about 2 millimeters. The desired thickness may be achieved by directly applying the thermochromic ink to the skin of a patient.

According to an alternative embodiment of the present invention, the thermo-sensitive hydrogel matrix itself can provide advantages for vein identification without a need to incorporate thermochromic additives. The thermosensitive hydrogel shows the dramatic change of color when its temperature approximates a certain programmed temperature, such as the lower critical solution temperature (LCST)—in other words, the temperature at which point hydrogel polymer chains shrink or contract and become opaque (i.e., white). Poly(N-isopropyl acrylamide) (pNIPAAm) gel, for example, appears transparent and in a swollen state at a temperature below LCST, and will manifest a dramatic change to being opaque and in a shrunken state at a temperature above LCST. This characteristic arises because the polymer chains in the gel network collapses and aggregates abruptly above LCST. The transition from transparent state to opaque state tends to be very fast, reversible, and easily detected, so this could be used as the visual indication of temperature change or difference. The transition temperature could be controlled by changing the formulation of gel preparation, for example, the addition of 0˜15% of alcohol (e.g. methanol, ethanol) in the formulation of gel preparation by the volume of total solution varies LCST of pNIPAAm gel in the range of 25˜32° C.

According to the invention, the thermosensitive hydrogel matrix is designed to exhibit an appropriate LCST behavior. The gel over the adjacent areas of veins does not show an observable change in appearance. The thermosensitive hydrogel matrix also provides the advantages like the cooling effect on skin for a greater temperature gradient and the intimate contact on skin for better performance.

According to certain embodiments, the hydrogel was prepared by dissolving agarose in water as 2 wt % and cooling in the mold, and one may then applies a thermo-chromic colorant can be gently spread on the top of the solidifying gel. The thermochromic colorant can be an ink which can be arranged as a layer by coating with a water-based slurry containing 49 wt % thermosensitive microcapsules from Matsui International Co. Inc. The microcapsules include a proton-accepting chromogen. In solution, the protonated form of the chromogen predominates at acidic pH levels (e.g., pH of about 4 or less). When the solution is made more alkaline through protonation, however, a color change occurs. One particularly suitable class of proton-accepting chromogens are leuco dyes, such as phthalides; phthalanes; acyl-leucomethylene compounds; fluoranes; spiropyranes; cumarins; and so forth. Exemplary fluoranes include, for instance, 3,3′-dimethoxyfluorane, 3,6-dimethoxyfluorane, 3,6-di-butoxyfluorane, 3-chloro-6-phenylamino-flourane, 3-diethylamino-6-dimethylfluorane, 3-diethylamino-6-methyl-7-chlorofluorane, and 3-diethyl-7,8-benzofluorane, 3,3′-bis-(p-dimethyl-aminophenyl)-7-phenylaminofluorane, 3-diethylamino-6-methyl-7-phenylamino-fluorane, 3-diethylamino-7-phenyl-aminofluorane, and 2-anilino-3-methyl-6-diethylamino-fluorane. Likewise, exemplary phthalides include 3,3′,3″-tris(p-dimethylamino-phenyl)phthalide, 3,3′-bis(p-dimethyl-aminophenyl)phthalide, 3,3-bis(p-diethylamino-phenyl)-6-dimethylamino-phthalide, 3-(4-diethylaminophenyl)-3-(1-ethyl-2-methylindol-3-yl)phthalide, and 3-(4-diethylamino-2-methyl)phenyl-3-(1,2-dimethylindol-3-yl)phthalide. Still other suitable chromogens are described in U.S. Pat. Nos. 4,620,941 to Yoshikawa et al.; 5,281,570 to Hasegawa et al.; 5,350,634 to Sumii et al.; and 5,527,385 to Sumii et al., which are incorporated herein in their entirety.

A desensitizer is also employed in the thermosensitive color-changing microcapsules to facilitate protonation of the chromogen at the desired temperature. More specifically, at a temperature below the melting point of the desensitizer, the chromogen generally possesses a first color (e.g., white). When the desensitizer is heated to its melting temperature, the chromogen becomes protonated, thereby resulting in a shift of the absorption maxima of the chromogen towards either the red (“bathochromic shift”) or blue end of the spectrum (“hypsochromic shift”). The nature of the color change depends on a variety of factors, including the type of proton-accepting chromogen utilized and the presence of any additional temperature-insensitive chromogens. The color change is typically reversible in that the chromogen deprotonates when cooled. Although any desensitizer may generally be employed in the present invention, it is typically desired that the desensitizer have a low volatility. For example, the desensitizer may have a boiling point of about 150° C. or higher, and in some embodiments, from about 170° C. to 280° C. Likewise, the melting temperature of the desensitizer is also typically from about 26° C. to about 34° C., and in some embodiments, from about 28° C. to about 33° C. Examples of suitable desensitizers may include saturated or unsaturated alcohols containing about 6 to 30 carbon atoms, such as octyl alcohol, dodecyl alcohol, lauryl alcohol, cetyl alcohol, myristyl alcohol, stearyl alcohol, behenyl alcohol, geraniol, etc.; esters of saturated or unsaturated alcohols containing about 6 to 30 carbon atoms, such as butyl stearate, lauryl laurate, lauryl stearate, stearyl laurate, methyl myristate, decyl myristate, lauryl myristate, butyl stearate, lauryl palmitate, decyl palmitate, palmitic acid glyceride, etc.; azomethines, such as benzylideneaniline, benzylidenelaurylamide, o-methoxybenzylidene laurylamine, benzylidene p-toluidine, p-cumylbenzylidene, etc.; amides, such as acetamide, stearamide, etc.; and so forth.

The color-changing microcapsules may also include a proton-donating agent (also referred to as a “color developer”) to facilitate the reversibility of the color change. Such proton-donating agents may include, for instance, phenols, azoles, organic acids, esters of organic acids, and salts of organic acids. Exemplary phenols may include phenylphenol, bisphenol A, cresol, resorcinol, chlorolucinol, β-naphthol, 1,5-dihydroxynaphthalene, pyrocatechol, pyrogallol, trimer of p-chlorophenol-formaldehyde condensate, etc. Exemplary azoles may include benzotriaoles, such as 5-chlorobenzotriazole, 4-laurylaminosulfobenzotriazole, 5-butylbenzotriazole, dibenzotriazole, 2-oxybenzotriazole, 5-ethoxycarbonylbenzotriazole, etc.; imidazoles, such as oxybenzimidazole, etc.; tetrazoles; and so forth. Exemplary organic acids may include aromatic carboxylic acids, such as salicylic acid, methylenebissalicylic acid, resorcylic acid, gallic acid, benzoic acid, p-oxybenzoic acid, pyromellitic acid, β-naphthoic acid, tannic acid, toluic acid, trimellitic acid, phthalic acid, terephthalic acid, anthranilic acid, etc.; aliphatic carboxylic acids, such as stearic acid, 1,2-hydroxystearic acid, tartaric acid, citric acid, oxalic acid, lauric acid, etc.; and so forth. Exemplary esters may include alkyl esters of aromatic carboxylic acids in which the alkyl moiety has 1 to 6 carbon atoms, such as butyl gallate, ethyl p-hydroxybenzoate, methyl salicylate, etc.

Encapsulation of the above-described components enhances the stability of the thermochromic ink during use. For example, the chromogen, desensitizer, developer, and other components may be mixed with a polymer resin (e.g., thermoset) according to any conventional method, such as interfacial polymerization, in-situ polymerization, etc. Suitable thermoset resins may include, for example, polyester resins, polyurethane resins, melamine resins, epoxy resins, diallyl phthalate resins, vinylester resins, and so forth. The resulting mixture may then be granulated and optionally coated with a hydrophilic macromolecular compound, such as alginic acid and salts thereof, carrageenan, pectin, gelatin and the like, semisynthetic macromolecular compounds such as methylcellulose, cationized starch, carboxymethylcellulose, carboxymethylated starch, vinyl polymers (e.g., polyvinyl alcohol), polyvinylpyrrolidone, polyacrylic acid, polyacrylamide, maleic acid copolymers, and so forth. The resulting microcapsules typically have a mean particle size of from about 5 nanometers to about 25 micrometers, in some embodiments from about 10 nanometers to about 10 micrometers, and in some embodiments, from about 50 nanometers to about 5 micrometers. Various other suitable encapsulation techniques are also described in U.S. Pat. Nos. 4,957,949 to Kamada et al.; 5,431,697 to Kamata et al.; and 6,863,720 to Kitagawa et al., which are incorporated herein in their entirety by reference thereto for all purposes. Commercially available encapsulated thermochromic substances may be obtained from Matsui Shikiso Chemical Co., Ltd. of Kyoto, Japan under the designation “Chromicolor” (e.g., Chromicolor AQ-Ink) or from Color Change Corporation of Streamwood, Ill. (e.g., black leuco powder having a transition of 33° C. or 41° C., red leuco powder having a transition of 28° C., yellow and red leuco powder having a transition of 31° C., or blue leuco powder having a transition of 33° C. or 36° C.).

The amount of the polymer resin(s) (e.g., thermoset) used to form the color-changing microcapsules may vary, but is typically from about 20 wt. % to about 80 wt. %, in some embodiments from about 30 wt. % to about 70 wt. %, and in some embodiments, from about 40 wt. % to about 60 wt. % of the microcapsules. The amount of the proton-accepting chromogen(s) employed may be from about 0.1 wt. % to about 20 wt. %, in some embodiments from about 0.5 wt. % to about 15 wt. %, and in some embodiments, from about 1 to about 10 wt. % of the microcapsules. The proton-donating agent(s) may constitute from about 0.5 to about 30 wt. %, in some embodiments from about 1 wt. % to about 20 wt. %, and in some embodiments, from about 2 wt. % to about 15 wt. % of the microcapsules. In addition, the desensitizer(s) may constitute from about 10 wt. % to about 70 wt. %, in some embodiments from about 15 wt. % to about 60 wt. %, and in some embodiments, from about 20 wt. % to about 50 wt. % of the microcapsules.

The nature and weight percentage of the components used in the thermosensitive color-changing microcapsules are generally selected so that the ink changes from one color to another color, from no color to a color, or from a color to no color at a desired activation temperature, which is generally from about 26° C. to about 34° C., and in some embodiments, from about 28° C. to about 33° C. in venous areas of the skin. However, the desired activation temperature may vary for different body parts. For example, the maximum skin temperatures normally observed over the veins in the antecubital fossa and upper forearm regions of the arm (e.g., inner bend of the arm), the most frequent sites for venipuncture, are about 32° C. at the examining room temperature (approximately 21° C. to 25° C.). Alternate sites for venipuncture include additional regions of the upper extremities (e.g., hands, wrists, and remaining forearm regions), where the maximum skin temperatures over veins are normally about 30° C., and the lower extremities (e.g., the feet and legs), where the maximum skin temperatures over the veins are normally about 28° C. In light of the above, the activation temperature may be tailored to the desired body part. For example, the activation temperature may be from about 30° C. to about 34° C., and in some embodiments, from about 31° C. to about 33° C. for the antecubital fossa region; from about 28° C. to about 32° C., and in some embodiments, from about 29° C. to about 31° C. for the upper extremities, and from about 26° C. to about 30° C., and in some embodiments, from about 27° C. to about 29° C. for the lower extremities.

According to other embodiments, in addition to a hydrogel matrix, the gel substrate may also contain other suitable cooling agents such as glycols (e.g., propylene glycol, butylene glycol, triethylene glycol, hexylene glycol, polyethylene glycols, ethoxydiglycol, and dipropyleneglycol); glycol ethers (e.g., methyl glycol ether, ethyl glycol ether, and isopropyl glycol ether); ethers (e.g., diethyl ether and tetrahydrofuran); alcohols (e.g., methanol, ethanol, n-propanol, iso-propanol, and butanol); triglycerides; ketones (e.g., acetone, methyl ethyl ketone, and methyl isobutyl ketone); esters (e.g., ethyl acetate, butyl acetate, diethylene glycol ether acetate, and methoxypropyl acetate); amides (e.g., dimethylformamide, dimethylacetamide, dimethylcaprylic/capric fatty acid amide and N-alkylpyrrolidones); nitriles (e.g., acetonitrile, propionitrile, butyronitrile and benzonitrile); sulfoxides or sulfones (e.g., dimethyl sulfoxide (DMSO) and sulfolane); and so forth. Certain solvents, such as organic solvents, may also have sanitizing and/or antimicrobial properties that can further reduce the risk of infection or contamination during venipuncture. For example, organic solvents, such as ethanol (enthalpy of vaporization of 38.6 kJ/mol) and methanol (enthalpy of vaporization of 37.4 kJ/mol), may be suitable cooling agents that also provide a sanitizing effect to the skin.

When employed, the total concentration of solvent(s) may vary, but is typically from about 30 wt. % to about 50 wt. %, in some embodiments from about 40 wt. % to about 50 wt. %, and in some embodiments, from about 50 wt. % to about 90 wt. % of the thermochromic ink. Likewise, the total concentration of the color changing microcapsules may range from about 1 wt. % to about 70 wt. %, in some embodiments from about 5 wt. % to about 60 wt. %, and in some embodiments, from about 10 wt. % to about 50 wt. %. Of course, the specific amount of solvent(s) employed depends in part on the desired solids content and/or viscosity of the thermochromic ink. For example, the solids content may range from about 0.01 wt. % to about 30 wt. %, in some embodiments from about 0.1 wt. % to about 25 wt. %, and in some embodiments, from about 0.5 wt. % to about 20 wt. %. By varying the solids content of the thermochromic ink, the presence of the color changing microcapsules may be controlled. For example, to form a thermochromic ink with a higher level of the microcapsules, the formulation may be provided with a relatively high solids content so that a greater percentage of the color-changing microcapsules are incorporated into the ink. In addition, the viscosity of the thermochromic ink may also vary depending on the application method and/or type of solvent employed. The viscosity is typically, however, from about 1 to about 200 Pascal-seconds, in some embodiments from about 5 to about 150 Pascal-seconds, and in some embodiments, from about 10 to about 100 Pascal-seconds, as measured with a Brookfield DV-1 viscometer using Spindle No. 18 operating at 12 rpm and 25° C. If desired, thickeners or other viscosity modifiers may be employed in the thermochromic ink to increase or decrease viscosity.

The thermochromic ink may also contain other components as is known in the art, such as a carrier (e.g., water) or co-carriers, such as lactam, N-methylpyrrolidone, N-methylacetamide, N-methylmorpholine-N-oxide, N,N-dimethylacetamide, N-methyl formamide, propyleneglycol-monomethylether, tetramethylene sulfone, tripropyleneglycolmonomethylether, propylene glycol, and triethanolamine (TEA). Humectants may also be utilized, such as ethylene glycol; diethylene glycol; glycerine; polyethylene glycol 200, 300, 400, and 600; propane 1,3 diol; propylene-glycolmonomethyl ethers, such as Dowanol PM (Gallade Chemical Inc., Santa Ana, Calif.); polyhydric alcohols; or combinations thereof. Further, additional temperature-insensitive chromogens may also be employed to help control the color that is observed during use of the thermochromic ink. Other additives may also be included to improve ink performance, such as a chelating agent to sequester metal ions that could become involved in chemical reactions over time, a corrosion inhibitor to help protect metal components of the printer or ink delivery system, and a surfactant to adjust the ink surface tension. Various other components for use in an ink, such as colorant stabilizers, photoinitiators, binders, surfactants, electrolytic salts, pH adjusters, etc., may be employed as described in U.S. Pat. Nos. 5,681,380 to Nohr et al. and 6,542,379 to Nohr et al., which are incorporated herein in their entirety by reference thereto for all purposes.

In certain embodiments, the polymeric gel substrate or pad also may be infused with topical antibiotics, antiseptic agents or disinfectants, or analgesic agents for pain, or a combination thereof. These agents can be topically or locally released when applied against mammalian skin. The term antiseptic refers to agents applied to the living tissues of humans, other animals, and plants in order to destroy (bactericidal) or inhibit the growth (bacteriostatic) of infectious microorganisms. Antiseptics are used in medical practice to prevent or combat bacterial infections of superficial tissues and to sterilize instruments and infected material. A distinction must be made between antiseptics and chemotherapeutic agents, such as antibiotics and sulfonamides, which are administered by mouth or by injection for the treatment of internal or generalized infections but may also be applied locally in the treatment or prevention of superficial infection. The major families of antiseptics are as follows. Alcohols are among the most widely used antiseptics, especially ethyl and isopropyl alchohol, which are commonly used in a 70 percent concentration with water. They are also widely used in combination with other antiseptic agents. The phenols contain a large number of common antiseptics and disinfectants, among them phenol (carbolic acid) and creosote, while such bisphenols as hexyl resorcinol and hexachlorophene are widely used as antiseptic agents in soaps. Chlorine and iodine are both extremely effective agents and can be used in high dilution. Hypochlorite solutions (e.g., Dakin's solution) are used in surgical practice. Iodine is an effective disinfectant of wounds, particularly when used in an alcohol solution. The quaternary ammonium compounds are more widely used as disinfectants than as antiseptics. Certain acridine dyes are used as antiseptics, as are some aromatic, or essential, oils.

Topical analgesia could be either added into the gel matrix or applied as a coating composition on the skin-contacting surface to lessen the pain of injection or puncture. Such compounds may include, for example, ibuprofen- or diclofenac-containing gel; capsaicin and or lidocaine. Salicylates can also be included in the composition as they decrease pain by reducing inflammation. Other examples of numbing agents that can be incorporated into the composition may be lanacane, benzocaine and premoxian. Hydro-cortisone could also be added to treat redness, itching, swelling or pain associated with various skin disorders.

### EXAMPLES

The following examples illustrate the use of embodiments of the present inventive device according to methods for visually locating blood vessel patterns.

I. Single Layer Hydrogel Matrix

The temperature sensitive substrate is formed of a single hydrogel matrix layer that has a thermochromic colorant mixed in the gel matrix. The colorant infused gel can regulate the local skin temperature. The overall thickness of the substrate gel matrix can be from about 0.1 mm to about 5 mm, and in some embodiments from about 0.5 mm to about 2 mm.

### Example 1

An amount of 0.4 g super agarose (OPTIMA USA, Inc.) was added to 20 g of distilled water and heated (˜80-90° C.) until agarose was completely dissolved. A water-based magenta thermochromic ink (Chromicolor AQ-Ink, type#25 with a temperature transition of 31° C., Matsui International Co. Inc., 49 wt % in water, 0.4 g) was added to the prepared agarose solution and stirred for complete mixing of dye and agarose. (The dye is believed to be suspended in the agarose solution.) The mixture was poured to a gel plate mold and allowed to set until the gel formed over night. The thickness of gel was controlled by changing the width of the space of gel plate mold (e.g., between ˜3 cm to ˜7 cm).

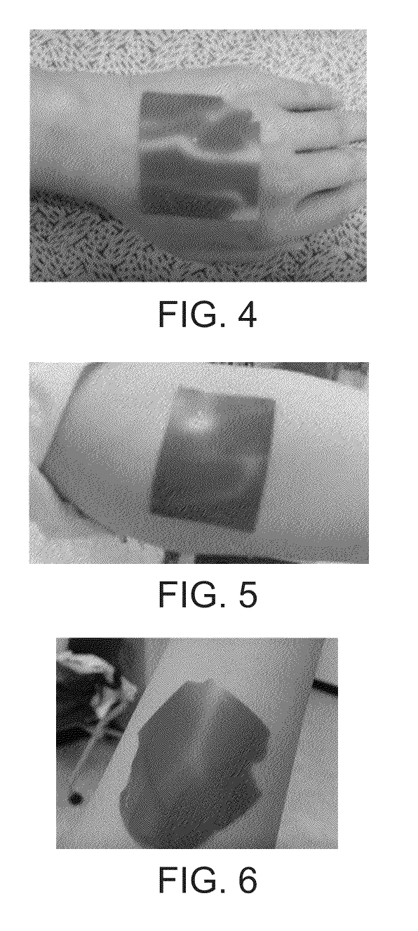

The purified gel system was put on the dorsal portion of a human hand, and the blood vessel identification was observed. The main channels of the dorsal venous network of the hand, formed by the dorsal metacarpal veins, was identified within a few seconds as clearly visible white lines as shown FIG. 4.

In another example, another purified gel substrate was put against the inside bend of the elbow, and the circulatory paths (radial and ulnar arteries, or medial cubital vein) are observed and identified within a few seconds of being applied as clear visibly contrasted as white lines against the darker gel pad background as shown FIG. 5. Another purified gel was put on the forearm and similar vein identification was observed. The vein was identified within a few seconds as a white line as clearly visible in FIG. 6. The hydrogel matrix also can be prepared by using other materials. For example, polyacrylamide or polyacrylate gels can be used for the hydrogel matrix. The details on preparation method are written in Example 2 and Example 3, respectively, as described below.

### Example 2

A 30% water-based solution of acylamide and bismethyleneacrylamide (29:1) (Bio-rad) was diluted to a 10% solution. A 0.5 g of thermochromic ink is added to a prepared monomer (5 mL) solution and completely mixed. Ammonium persulfate (Sigma) was prepared as 10% water-based solution for the initiator. 50 μl of initiator solution and 5 μl of tetramethylethyleneamine (Sigma) in solution was added to the mixture of monomer and thermochromic dye solution and slightly stirred for complete mixing. The mixture immediately was poured to the gel plate and cured for 3 hrs. at room temperature. The prepared gel was separated from the gel plate and purified with distilled water to extract non-reacted monomers for two days.

### Example 3

An amount of 1.45 g hydroxyethyl methacrylate and 0.05 g polyethyleneglycol-diacrylate (mixed as cross-linker in the solution) was dissolved in 5 ml of distilled water. 0.5 g of thermochromic dye was added to the monomer solution and completely mixed. Ammonium persulfate was prepared as 10% water-based solution for the initiator. 50 μl of initiator solution and 5 μl of tetramethylethyleneamine was added to the mixture of monomer and thermochromic dye solution and slightly stirred for complete mixing. The mixture was poured to the gel plate mold and cured for 3 hrs. at room temperature. The prepared gel was separated from the gel plate and purified with distilled water to extract non-reacted monomers for two days.

### Example 4

In an alternate embodiment, a polymeric matrix or gel pad is composed of a thermosensitive gel of NIPAM (not containing thermochromic dye). The pNIPAAm gel pad was synthesized and purified according to following steps: About 1.43 g of N-isopropylacrylamide (Sigma) was completely dissolved in 5 ml of water/methanol (10/1 volume ratio) mixture. 0.2 g of N,N′-methylenebisacrylamide (Sigma) was added as a crosslinking agent. An amount of 0.2 g ammonium persulfate (Sigma) was dissolved in 0.5 ml of distilled water and added into the prepared monomer solution. The polymerization was carried out in the glass gel plate after adding 5 μl of N,N,N′,N′-tetramethylethylenediamine (Sigma) at room temperature (20° C.) for 24 hours. The prepared gel substrate was separated from the gel plate and purified with distilled water to extract non-reacted monomers for a period of two days.

II. Two-Layer Hydrogel Matrix

A two layer temperature-sensitive substrate can be composed of a thin temperature sensing colorant coating or layer deposited on the skin-contacting surface of a translucent or clear hydrogel matrix. When the substrate is applied against a patient's skin, the thermochromic colorant is oriented against the skin surface and underneath the transparent gel. This type of configuration affords some manufacturing advantages by reducing the total amount of thermochromic colorant used, The thickness of the temperature sensing colorant layer can be from about 0.05 mm to about 2 mm, and in some embodiments from about 0.1 mm to about 1 mm. The thickness of the clear gel is can be from about 0.5 mm to about 3 mm, and in some embodiments form about from 0.75 mm to 1 mm.

### Example 5

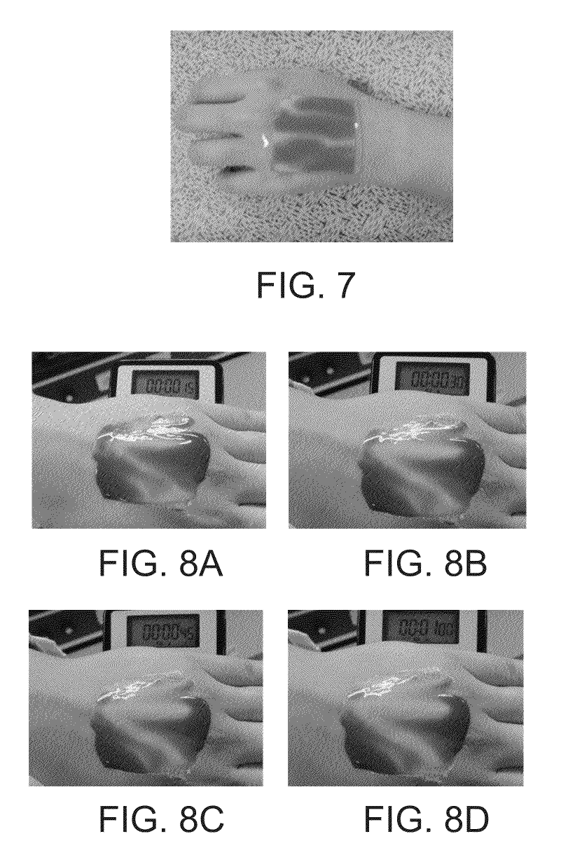

In a two-layered embodiment (i.e., a clear gel layer and a thermochromic layer), a thermochromic dye solution is prepared as a temperature sensing layer in a gel plate mold, and an agarose-water solution (2 wt % of agarose completely dissolved as in Example 1, above) was poured over the temperature sensing layer and allowed to set until gel formation (FIG. 2). The purified gel substrate was put on the back of hand and the vein identification was observed as shown in FIG. 7. The gel in the skin surface directly over the vein became opaque within a few seconds, while the gel in the areas adjacent to veins remained transparent for a longer duration of time. In comparing the two embodiments, the present example and Example 1, one does not observe big functional differences between the two types, because the hydrogel matrix modulates or controls the heat transfer rate. These self-cooling substrate examples both exhibit comparably good temperature sensitivity and generally clear imaging of the main blood vessels. The two-layered substrate can be fabricated with a lesser amount of thermochromic pigment than used in the single-layer gel suspension by at least 10-20%.

### Comparative Example

Thermochromic ink is provided as a water-based slurry containing 49 wt % of thermosensitive dye microcapsules from Matsui International Co. Inc. The thermochromic ink, without gel substrate, is directly and gently spread over the dorsal surface or top of a human hand and the development of the temperature differentiated images of underlying blood vessels is captured over time in the series of photos in FIGS. 8A-8D. The image of the blood vessel appeared within about 15 seconds; however, the identified veins did not appear clearly. Without cooling from a gel polymer matrix, in a short time from initial application, the temperature differentiated images of the veins appeared to be wider or thicker than actual size. This is due to the low temperature differential gradient between the blood vessels and surrounding tissues, when the initial evaporative effect lessened or dissipated. The temperature gradient between the skin surface and the thermochromic ink directly over the vein and adjacent areas of the skin began to recover to original temperature within about one minute. Hence, the ability to clearly image and identify veins disappeared before the water in the ink even dried.

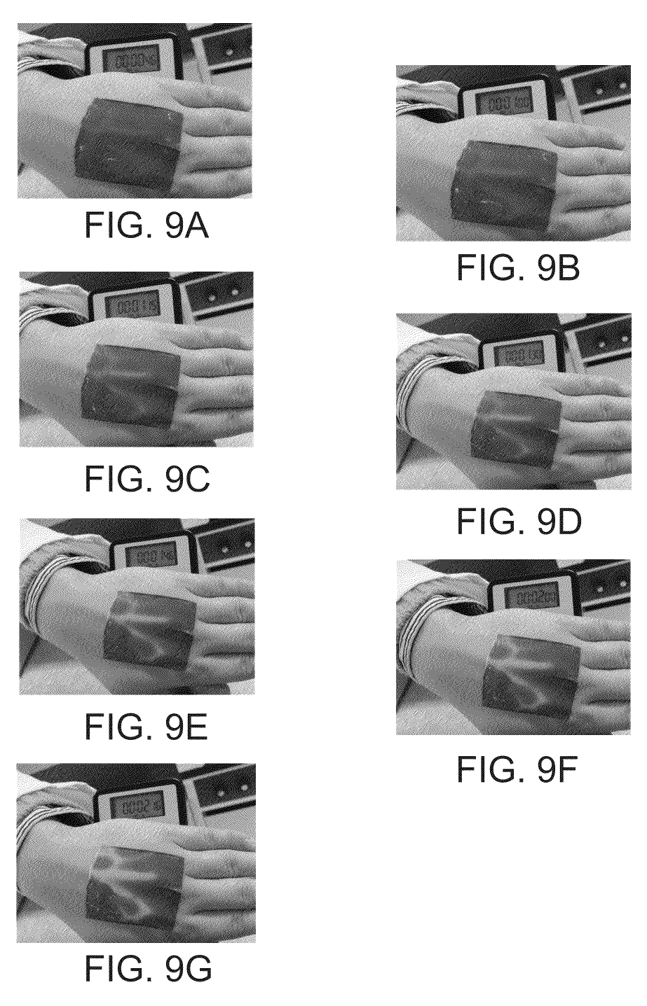

In contrast, according to the present invention, the same thermochromic ink described above is applied with a hydrogel matrix. The hydrogel is prepared by dissolving agarose in water as 2 wt % and cooling in the gel-forming mold. For the self-cooling gel pad, the thermochromic ink is gently spread onto the surface of the hydrogel and the solvent over the top of gel is allowed to slightly dry for the completion of coating. The gel pad is again placed on the dorsal surface of a human hand, with the thermosensitive dye-colored side facing toward the skin, and the temperature differentiated images are captured over time. The image of the underlying blood vessels began to develop within about 45-50 seconds, and appeared relatively sharp and clearly identified over a period of about 60-150 seconds. The image remained relatively sharp over about 45-70 seconds as shown FIGS. 9A-9G.

The present invention has been described both generally and in detail by way of examples and the figures. Persons skilled in the art, however, can appreciate that the invention is not limited necessarily to the embodiments specifically disclosed, but that substitutions, modifications, and variations may be made to the present invention and its uses without departing from the spirit and scope of the invention. Therefore, changes should be construed as included herein unless the modifications otherwise depart from the scope of the present invention as defined in the following claims.

We claim:

1. A temperature-sensitive substrate, the substrate comprising a first major surface and a second major surface, said substrate formed from a self-cooling, polymeric matrix having at least a thermochromic colorant, ink, or dye that is either a) admixed within said matrix or b) forming a layer on one of said major surfaces, said thermochromic colorant, ink, or dye having a color-change sensitivity at a temperature in a range between about 35.0° C. to about 40.5° C.

2. The temperature-sensitive substrate according to claim 1, wherein said substrate has a plurality of pores distributed over said substrate, each of said pores traversing from said first surface to said second surface of said substrate.

3. The temperature-sensitive substrate according to claim 2, wherein said plurality of pores are adapted to accommodate the width of a syringe needle or cannula.

4. The temperature-sensitive substrate according to claim 2, wherein said plurality of pores are distributed in a predetermined pattern over said substrate.

5. The temperature-sensitive substrate according to claim 1, wherein said self-cooling polymeric gel is a hydrogel matrix.

6. The temperature-sensitive substrate according to claim 5, wherein said hydrogel matrix evaporates water when applied against a source of heat.

7. The temperature-sensitive substrate according to claim 1, wherein said thermo-chromic colorant, ink, or dye comprises microcapsules containing one of the following: a proton-accepting chromogen, or liquid crystal or fatty acid derivatives of cholesterol system.

8. The temperature-sensitive substrate according to claim 1, wherein said substrate has a thickness from about 0.1 mm up to about 7 mm.

9. The temperature-sensitive substrate according to claim 8, wherein said substrate has a thickness between about 0.25 and about 3 mm.

10. The temperature-sensitive substrate according to claim 1, wherein said substrate has a composition that comprises pigment:water ratio that ranges from 0.25-10:100 by weight.

11. The temperature-sensitive substrate according to claim 10, wherein said ratio of pigment:water is 0.5-5:100 by weight.

12. The temperature-sensitive substrate according to claim 1, wherein said substrate has an antimicrobial coating on at least one of said major substrate surfaces or distributed homogeneously throughout said substrate.

13. The temperature-sensitive substrate according to claim 1, wherein said substrate is self-tacking when applied against mammalian skin.

14. The temperature-sensitive substrate according to claim 1, wherein said polymeric gel has an antiseptic agent, pain analgesic agent, or a combination thereof that is topically or locally released when applied against mammalian skin.

15. The temperature-sensitive substrate according to claim 14, wherein said antiseptic agent or pain analgesic is in a layer coating a major surface of said substrate that contacts against mammalian skin.

16. A thermal-imaging article comprising: a substrate formed of a self-cooling hydrogel polymer matrix, a plurality of pores that extend through said substrate from a first major surface to a second major surface, said substrate having at least a thermochromic colorant that is either a) admixed within said hydrogel polymer matrix or b) coating one of said major surfaces, said substrate has an antiseptic agent, pain analgesic agent, or a combination thereof that is topically or locally released when applied against mammalian skin.

17. The thermal-imaging article according to claim 16, wherein said substrate has a composition that comprises pigment:water ratio that ranges from 0.25-10:100 by weight.

18. The thermal-imaging article according to claim 16, wherein said substrate is self-tacking when applied against mammalian skin.

19. The thermal-imaging article according to claim 16, wherein said substrate is an aid for identifying the relative locations of blood vessels immediately under skin surface.

20. A method for thermal-imaging body regions with greater localized temperatures than surrounding tissues, the method comprising: providing a self-cooling polymer gel substrate containing a thermo-chromic colorant; applying said gel substrate to a heat-emitting mammalian body; observing a color contrast that develops from a temperature gradient between areas of said gel substrate that cover warmer body regions and adjacent cooler body regions.

21. The method according to claim 20, further comprising applying said self-cooling polymer gel substrate to areas of said mammalian body where underlying blood vessels are within about 1-3 cm of tissue or skin surface.

22. The method according to claim 20, wherein said temperature difference between body regions and surrounding tissues is about 0.1-2° C. or greater.

23. The method according to claim 20, wherein said body region has a cancer growth.

24. A method for locating a blood vessel, the method comprising: providing a self-cooling hydrogel polymer substrate containing a thermochromic colorant that changes color at a temperature in a range between about 35.0° C. to about 40.5° C.; placing said hydrogel polymer substrate against bare skin of a portion of a body over which blood vessels may be accessed; and observing a color contrast develop that arises from a temperature gradient between areas of said gel substrate that cover warmer body regions and adjacent cooler body regions.

25. The method according to claim 24, further comprising inserting a hypodermic needle or cannula into a blood vessel that is thermal-imaged by said hydrogel polymer substrate.

26. The method according to claim 24, wherein said needle is inserted through a pore which traverses from a first surface to a second surface of said hydrogel polymer substrate.

27. The method according to claim 24, wherein when placed against said bare skin surface, said polymer substrate prolongs the difference in relative temperature gradient between blood vessels and surrounding tissues for up to about 6 or 7 minutes.
